(19) United States
(12) Patent Application Publication
(10) Pub. No.: US 2017/0113197 A1
(43) Pub. Date: Apr. 27, 2017

(54) Drive System for Static Batch Reactor

(71) Applicant: Anaero Technology Ltd, Cambridge (GB)

(72) Inventors: Ray Middleton, Cambridge (GB); Edgar Blanco, Cambridge (GB); Robin Proctor, Manchester (GB)

(21) Appl. No.: 15/317,264

(22) PCT Filed: Jun. 8, 2015
(86) PCT No.: PCT/GB2015/051658

§ 371 Date: Dec. 8, 2016

(30) Foreign Application Priority Data
Jun. 9, 2014 (GB) 1410204.0

(51) Int. Cl. B01F 7/16; B01F 13/10; F16H 57/031; B01J 19/18; G01N 33/00; F16H 1/22; B01F 7/00; B01F 15/00

(52) U.S. Cl. CPC B01F 7/1695; B01F 7/00166; B01F 7/00725; B01F 13/1022; B01F 15/00454; B01F 15/00136; B01J 19/1856; G01N 33/0027; F16H 1/22; F16H 57/031; B01F 2015/00603; B01F 2215/0037; B01J 2219/00164

(57) ABSTRACT

A device for driving a plurality of rotatable agitators for use in a static batch reactor system, a reactor bottle cap for use with the rotatable agitator driving device and a static batch reactor system which includes the rotatable agitator driving device and bottles fitted with the reactor bottle cap are disclosed. The rotatable agitator driving device includes a planar case having first and second opposite faces, a plurality of drive heads projecting from the case through the second face and a gear train protectively housed in the case. The gear train includes a driver gear having a coupling for receiving drive from a motor, a set of idler gears and a set of driven gears. Each driven gear attached to a respective drive head for driving a rotatable agitator. The gear train is configured to cause the drive heads to rotate at the same rate.

### FIELD OF THE INVENTION

The present invention relates to a device for driving a plurality of rotatable agitators for use in a static batch reactor system, to a reactor bottle cap for use with the rotatable agitator driving device and to a static batch reactor system which includes the rotatable agitators driving device and bottles fitted with the reactor bottle caps.

### BACKGROUND

Anaerobic digestion can be used to process biodegradable waste, such as waste food and/or sewage, to produce a biogas which includes methane. Before committing resources to building a large-scale anaerobic digestion system, experiments and laboratory-scale reactors are used to simulate an anaerobic digestion process.

Laboratory-based residual biogas potential (RBP) (which is also referred to as “biochemical methane potential (BMP)”) tests are used to identify whether a feedstock is suitable for anaerobic digestion and, if so, to identify its potential yield.

These tests are usually carried out in batches using an array of static bioreactors. An example of an RBP test system is the AMPTS II Automatic Methane Potential Test System marketed by Bioprocess Control AB, Lund, Sweden.

The AMPTS II system comprises 15 glass reactors which sit in a thermostatically-controlled water bath. Each reactor is provided with an electric motor which drives a mechanical agitator (or “stirrer”).

### SUMMARY

According to a first aspect of the present invention there is provided a device for driving a plurality of rotatable agitators for use in a static batch reactor system. The device comprises a planar case having first and second opposite faces, a plurality of drive heads projecting from the case through the second face and a gear train protectively housed in the case. The gear train comprises a driver gear having a coupling for receiving drive from a motor, a set of idler gears and a set of driven gears, each driven gear attached to a respective drive head for driving a rotatable agitator. The gear train is configured to cause the drive heads to rotate at the same rate.

This can help to ensure consistent agitation in different reactors in a static batch reactor system. The case can also help to avoid clothing or a part of the body (such as finger) being pulled into the gear train.

The gear train may be configured to cause the drive heads to rotate in the same angular direction.

There may be at least four drive heads, at least six drive heads, at least eight drive heads or at least at least nine drive heads. There may be no more than twenty-four drive heads, no more than twenty drive heads, no more than eighteen drive heads, no more than sixteen drive heads, no more than fifteen drive heads, no more than twelve drive heads or no more than ten drive heads. There may be six, eight, nine, ten, twelve, fourteen, fifteen, sixteen, eighteen, twenty or twenty four drive heads.

The drive heads may be arranged in an array. The array may be a rectangular array. The drive heads may comprise at least three drive heads arranged spaced along a first direction and at least three drive heads spaced along a second orthogonal direction. The array may be a hexagonal array.

The case may comprises a main portion having a main recess for accommodating the receiving the gears and further recesses for accommodating shafts on a first face of the gears and a lid portion having recesses for accommodating shafts on a second, opposite face of the gears.

The device may further comprise one or more supports for fixing the device to a bath.

The device may further comprise a motor coupled to the driver gear. The motor may be a variable speed motor. The motor may be a single-drive motor. The motor may be a slow-speed geared motor having an output of 10 to 300 rpm. The motor may be disposed on the first face of the case.

Each drive head may includes a seat for receiving a drive shaft of a rotatable agitator. Each seat may comprise a conical recess and a centrally-aligned axially-extending slot.

The present invention also seeks to provide an improved cap for a static reactor bottle.

According to a second aspect of the present invention there is provided a cap for a static reactor bottle. The cap comprises a main body comprising a top portion having a centre and a periphery and an annular skirt depending downwardly from the periphery of the top portion having an internal screw thread for engaging an external screw thread of a bottle. The cap comprises an elongate neck extending upwardly from the centre of the top portion along a central axis to a first distal end having an axial recess. The cap comprises an elongate insert depending downwardly from the top potion of the main body along the central axis to a second, opposite distal end. The cap comprises a passage extending along the central axis from the axial recess to the second distal end though the neck, main body and insert, the passage including at a first section which is relatively narrow and a second section disposed between the first section and the recess which is relatively wide. The cap comprises a duct extending through the top portion from a pipe connector on a top side of the top portion to an underside.

When engaged with a bottle, the cap can allow the contents of the bottle to be mixed without the atmosphere inside the bottle being contaminated by the atmosphere outside the bottle and vice versa. The long passage can help to discourage or prevent liquid content (which is under pressure from gas in the headspace above the liquid) from migrating up the shaft of the agitator and escaping.

The axial recess can be used to provide a gas seal. In particular, a small amount of water or viscous inert liquid can be placed in the axial recess to provide a barrier against contamination from the atmosphere outside the bottle via the shaft of agitator.

The insert may be removably attached to the main portion.

The duct may be inclined with respect to the central axis.

The cap may further comprise a further passage extending through the top portion from the underside to the topside. The further passage may be use to insert a tube (or “port”) into the bottle. The further passage may run parallel to the passage.

The cap may further comprise a tube passing through or in fluid communication with the further passage which depends downwardly from the main body of the cap.

When the end of tube passes below the surface of the liquid contents, the tube can allow access to the liquid contents in the bottle while minimising or preventing contamination with air outside the bottle. The tube can be used, for example, for conducting tests, such pH measurements or electrical conductivity monitoring, for supplementation (of, for instance, trace elements), for re-inoculation, and/or for starting a test.

According to a third aspect of the present invention there is provided a cap assembly for a static reactor bottle. The cap assembly comprises a cap and an agitator comprising a shaft disposed in the passage and paddle.

The tube has a distal end which may lie at a level at or below a mid-point between top and bottom of the inside of the bottle.

According to a fourth aspect of the present invention there is provided a bottle assembly comprising a bottle comprising a body and a neck having an external screw thread and a cap assembly which is screw engaged with the bottle.

The cap may comprise a main body comprising a top portion having a centre and a periphery and an annular skirt depending downwardly from the periphery of the top portion having an internal screw thread for engaging an external screw thread of a bottle, an elongate neck extending upwardly from the centre of the top portion along a central axis to a first distal end having an axial recess, an elongate insert depending downwardly from the top potion of the main body along the central axis to a second, opposite distal end, a passage extending along the central axis from the axial recess to the second distal end though the neck, main body and insert, the passage including at a first section which is relatively narrow and a second section disposed between the first section and the recess which is relatively wide, a duct extending through the top portion from a pipe connector on a topside of the top portion to an underside and an agitator comprising a shaft disposed in the passage and paddle.

The bottle may have a wide neck opening, i.e. a ratio of neck inner diameter to body outer diameter of at least 0.5 or at least 0.6.

The bottle may have a volume of at least 500 millilitres, at least 1 litre or at least 2 litres. The bottle may have a volume of no more than 5 litres, no more than 2 litres or no more than 1 litre. The bottle may have a neck inner diameter of at least 50 mm or at least 100 mm.

According to a fifth aspect of the present invention there is provided a static batch reactor system comprising a water bath, at least two bottle assemblies at least partially immersed in the water bath and a driving device, wherein each agitator is coupled to a respective drive head.

The system may further comprise at least one gas flow measuring device, each pipe connector in fluid communication with a respective gas flow measuring device.

The system may be arranged for testing a biological process, which may be aerobic, anoxic or anaerobic, such as residual biogas potential, biomethane potential or toxicity assays.

According to a sixth aspect of the present invention there is provided a method comprising adding feedstock to at least two bottles, assembling at least two bottle assemblies; placing the at least two bottle assemblies in a bath; fitting an agitator driving device, wherein each agitator is coupled to a respective drive head and applying power to a motor coupled to the driver gear.

The method may further comprise providing a flow sensor for each bottle assemblies.

According to a seventh aspect of the present invention there is provided a method using the static batch reactor system, the method comprising driving the device.

### BRIEF DESCRIPTION OF THE DRAWINGS

Certain embodiments of the present invention will now be described, by way of example, with reference to the accompanying drawings, in which:

FIG. 1 is a perspective view of a static batch reactor system which includes bottles, bottle cap assemblies having agitators, an agitator driving device and motor;

FIG. 2 is a perspective view of the static batch reactor system shown in FIG. 1 without the agitator driving device;

FIG. 3 is a partial cross sectional view of a portion of static batch reactor system shown in FIG. 1;

FIG. 4 is a perspective view of a cap assembly which includes a cap and an agitator;

FIG. 5 is a perspective view of a bottle assembly which includes a cap assembly and a bottle;

FIG. 6 is a plan view of a main body of a cap;

FIG. 7 is cross-sectional view of a main body and neck of a cap shown in FIG. 6 taken along the line A-A;

FIG. 8 is cross-sectional view of a paddle shaft guide tube;

FIG. 9 is a perspective view from above of the agitator driving device and motor shown in FIG. 1;

FIG. 10 is a partial side view of the agitator driving device shown in FIG. 9;

FIG. 11 is a perspective view from below of the agitator driving device shown in FIG. 1; and

FIG. 12 is a schematic block diagram of a residual biogas potential (RBP) testing system.

### DETAILED DESCRIPTION OF CERTAIN EMBODIMENTS

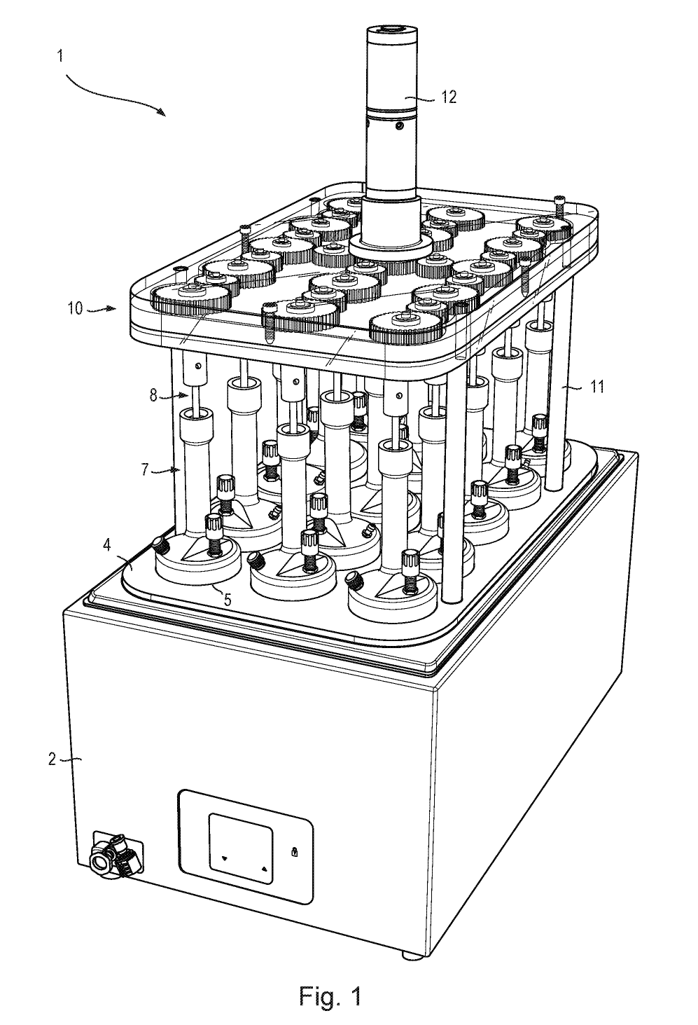

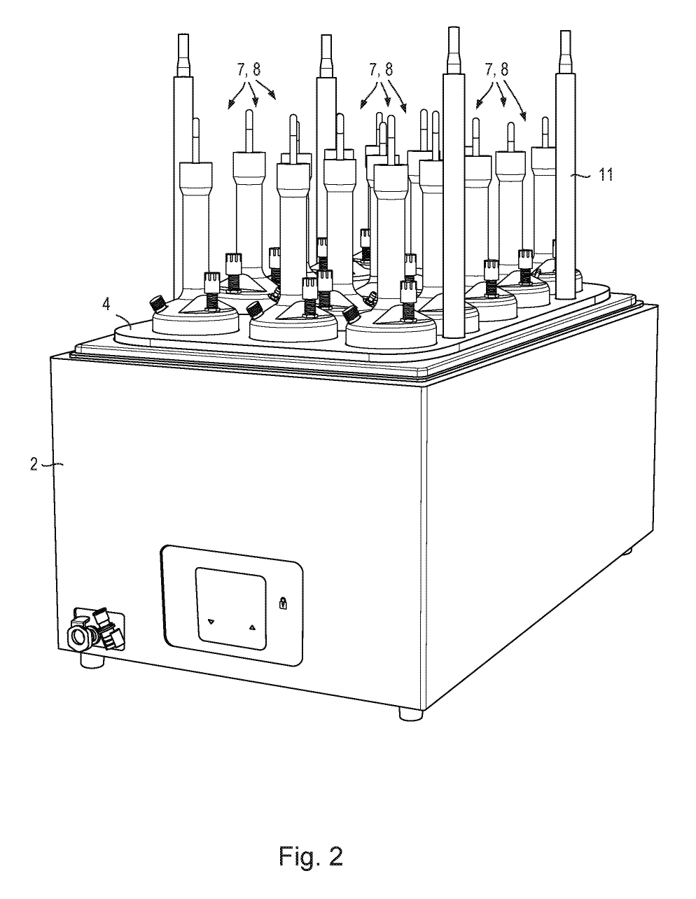

Referring to FIGS. 1, 2 and 3, a static batch rector system 1 is shown.

The system 1 can be used for residual biogas potential (RBP) testing.

The system 1 includes a water bath 2 filled with water 3, a water bath lid 4, formed from polymethylmethacrylate or other suitable plastics material, having an array of apertures 5 for accommodating reactor bottles 6, one or more reactor bottles 6, each bottle 6 provided with a long-necked cap 7 which guide rotatable agitators 8 (or “paddles”) for stirring a liquid 9, e.g. digestate, in the bottle 6 and an agitator driving device 10 which is held above the water bath 2 by a set of posts 11. The agitator device 10 is coupled to a variable-speed electric motor 12 which drives the agitator driving device 10 which, in turn, drives the agitators 8. The liquid may take the form of a mixture which can include liquid(s) and/or solid(s). The liquid may take the form of a semi-solid, a suspension, an emulsion or other mixtures.

In this case, the system 1 can accommodate up to fifteen reactor bottles 6 in fifteen available sites, although one or more sites may be left empty. The system 1 may have more or fewer available sites.

Each bottle 6 each has a capacity of 1 litre. However, smaller or larger bottles 6 can be used. Each bottle 6 has a wide neck 13. In particular, the neck 13 has an inner diameter, d₁, which is at least half an outer diameter, d₂, of the bottle 6. Thus, the bottle 6 can accommodate an agitator 8 having a wide, i.e. large-radius, mixing paddle 14. The neck 13 has an external screw thread 15 which allows the cap 7 to be screw-engaged with the bottle 6. The bottles 6 stand in the water bath 2 on a platform 16.

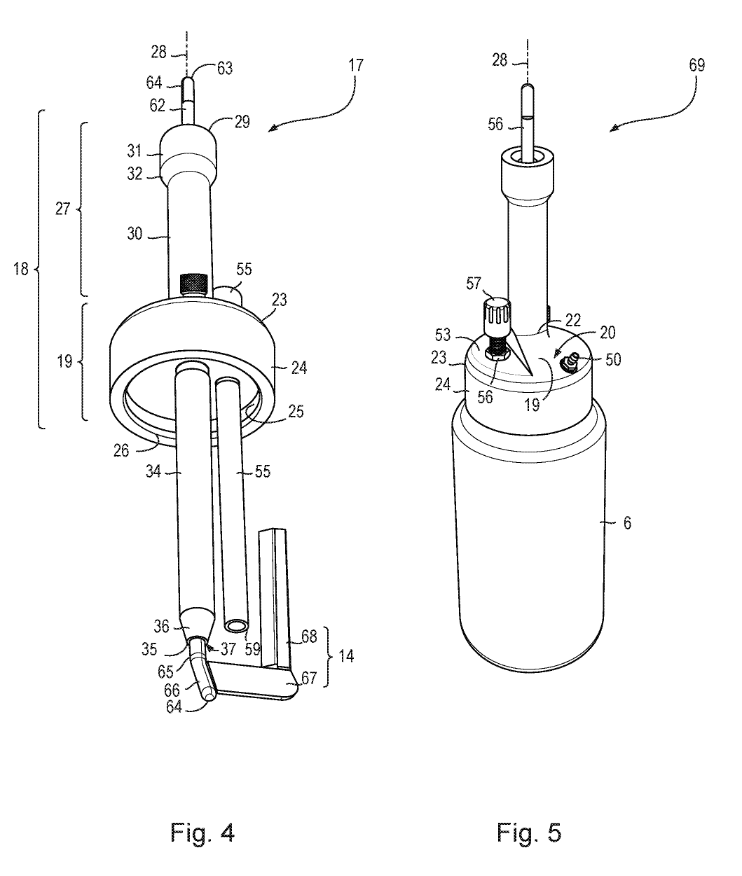

FIG. 4 shows a cap assembly 17 which consists mainly of a cap 7 and an agitator 8. The cap 7 is formed from polyoxymethylene (or “acetal”). However, other suitable plastic materials may be used, for example, plastic materials which are strong and hard enough to resist wear and use.

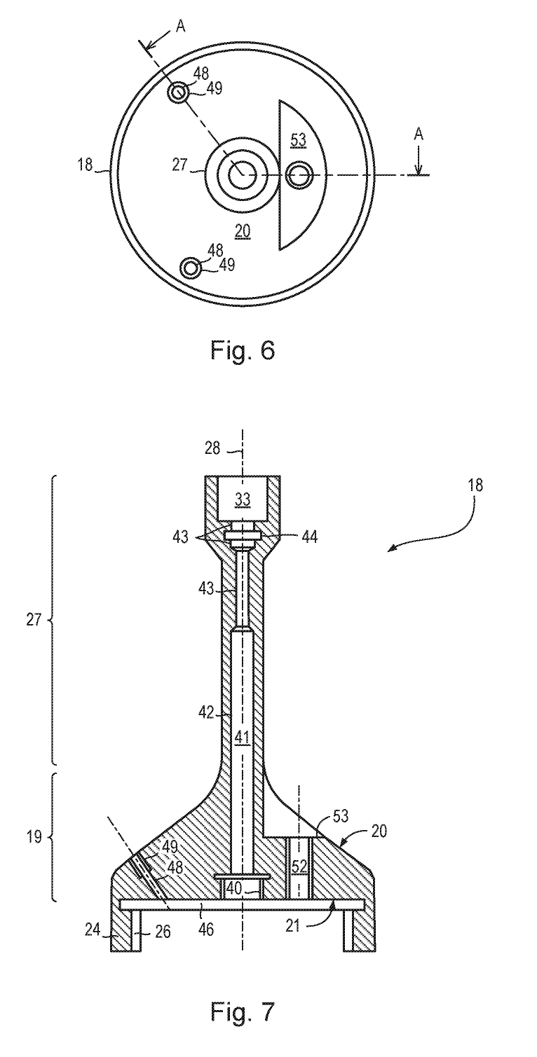

Referring to FIGS. 4, 5, 6 and 7, each cap 7 has a main body 18. The main body 18 includes a generally frustoconical top (or “cover”) portion 19 (best shown in FIGS. 5 and 7) having a top surface 20 and an underside 21. The top portion 19 has a centre 22 and a periphery 23. An annular skirt 24 depends downwardly from the periphery 23 of the top portion 19. An inner wall 25 of the skirt 24 has a screw thread 26 for engaging the screw thread 15 of a bottle 6.

The main body 18 also includes an elongate neck 27 (herein also referred to as a “stem”) extending upwardly from the centre 22 of the top portion 19, along a central axis 28, to a distal end 29. The neck 27 has a lower, long narrow section 30 and an upper, short wide section 31 joined by a sloping section 32. The neck 27 has an axial recess 33 its distal end 29.

In this example, the main body 18 has a total length of about 155 mm and an outer diameter of 74 mm. However, the dimensions can be varied according to the size of the bottles 6 and other components of the system 1.

The cap 7 has a paddle shaft guide tube 34 (herein also referred to as an “elongate insert”) which depends downwardly from the top potion 19 of the main body 18, along the central axis 28, to a second, opposite distal end 35 having a short taper 36 and an orifice 37.

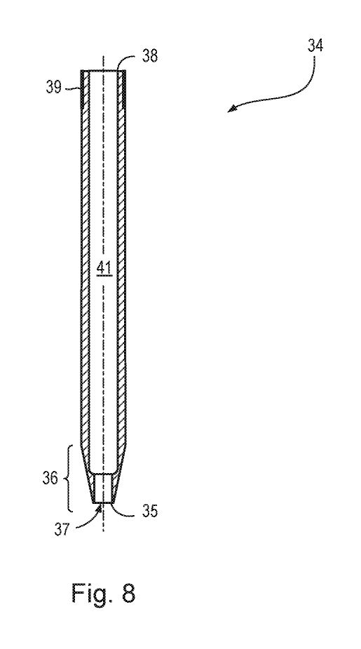

Referring also to FIG. 8, the tube 34 is separate piece having, at a proximal end 38, an outer screw thread 39 which is screw-engageable with an inner screw thread 40 in the underside 21 of the top portion 19 of the main body 18.

In this example, the guide tube 34 (including the threaded section) has a length of about 150 mm and an outer diameter of 15 mm. However, the dimensions can be varied according to the size of the bottle 6 and the agitator 8.

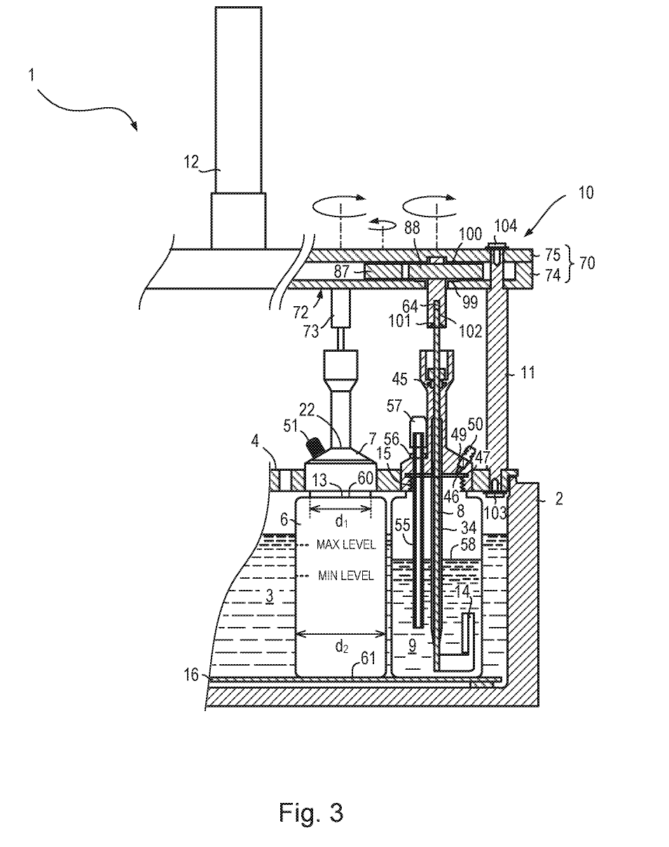

A passage 41 extends from the axial recess 33 along the central axis 28 to the second distal end 35 though the neck 27, main body 18 and guide tube 34. The passage 41 includes a first section 42 which is relatively wide (in this case, about 11 mm) and a second section 43 disposed between the first section 42 and the recess 33 which is relatively narrow (in this case, about 6 mm). A short, third section 44 is provided between the recess 33 and the second section 43 which has an intermediate diameter (in this case, about 7.5 mm) and which is provided with an annular seal groove 44 (in this case, having a diameter of about 11 mm) for accommodating a washer 45 (FIG. 3).

The annular skirt 24 includes an annular seal groove 46 between the top portion 19 of the main body 18 and the screw thread 26 of the annular skirt 24 for accommodating a washer 47.

The cap 7 includes first and second gas feed holes (or “ducts”) 48 which are generally drilled square to the top surface 20 and tapped to provide threaded sections 49. The holes 48 extend through the top section 19 from the top surface 20 to the underside 21. Pipe connectors 50 in the form of ribbed pipe fittings are fitted in the threaded sections 49. The pipe connectors 50 can be provided with a cap 51.

The cap 7 also includes a further passage 52 (herein referred to as a “test port”) extending from a recessed ledge 53 cut in the top section 19 of the main body 18 to the underside 21. The test port 52 is threaded. The test port 52 is used to insert a tube 55 into a bottle 6 which can be used for sampling, conducting tests (such as a pH measurement), supplementation and the like. The test port analysis tube 55 is held in place by a locking nut 56 and is provided with a cap 57.

The tube 55 is sufficiently long to drop below the surface 58 of the liquid 3 in the bottle 6. The tube 55 has a distal end 59 which lies between top 60 (for example, at the foot of the neck 13) and bottom 61 of the inside of the bottle 6, preferably at or below a mid-point between the top and bottom 60, 61. The distance between the distal end 59 and the bottom of the bottle 61 can be between 0.1 h and 0.5 h, where h is the height between the top 60 and bottom 61 of the bottle 6. However, the length of tube and/or its position is (are) such that the tube does not interfere with rotation of the agitator 8.

The agitator 8 includes an elongate shaft 62 extending between first and second ends 63, 64. The first end 63 has a rectangular tongue drive tip 64. The shaft 62 includes an elbow 65 such that the end portion 66 of the shaft 62 between the elbow 65 and the end of the shaft 64 is inclined with respect to the central axis 28. The end portion 66 supports the paddle 14 which includes a planar wing portion 67 and an upwardly extending thin blade portion 68.

A small amount of water or viscous inert liquid (not shown) can be placed in the axial recess 33 to provide a barrier against contamination from the atmosphere outside the bottle via the shaft 64 of agitator 8.

As shown in FIG. 5, the cap 17 is screw engaged with the bottle 6 to form a bottle assembly 69.

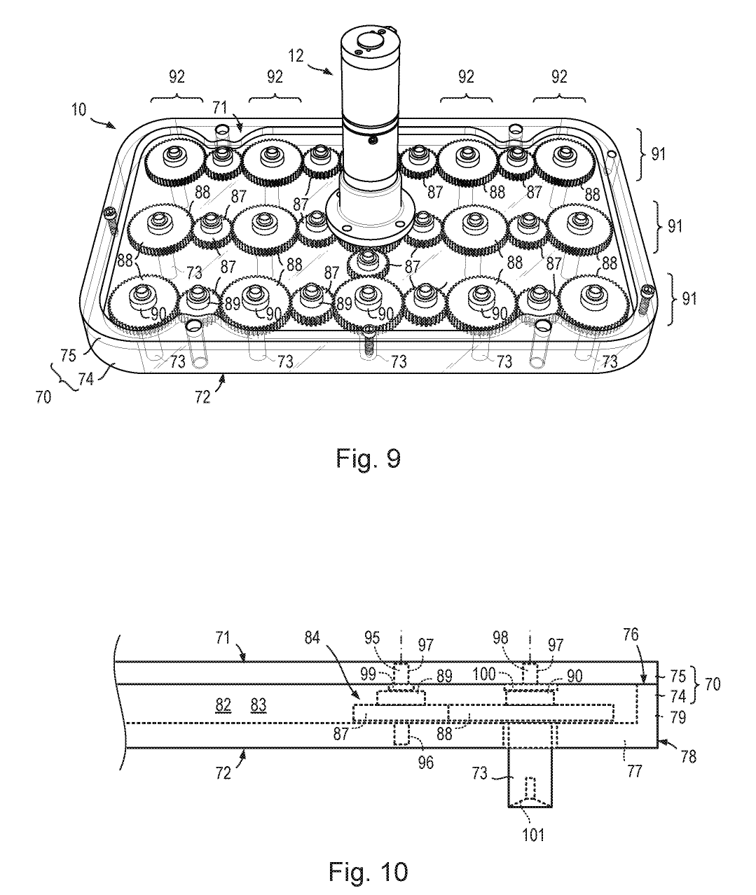

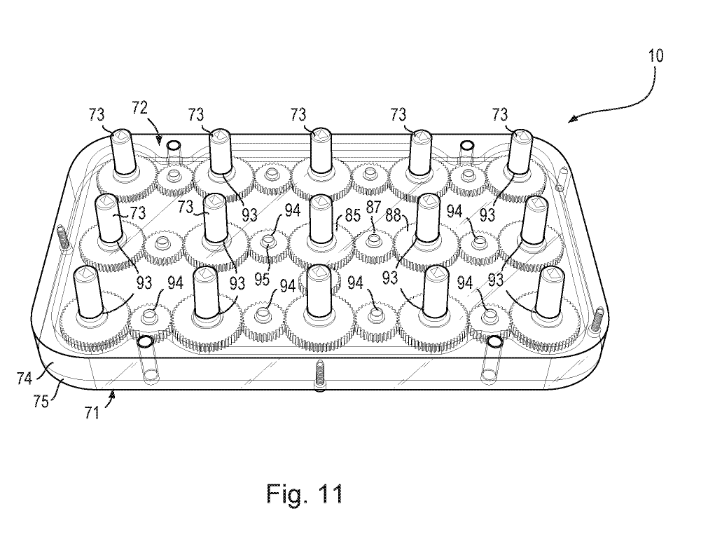

Referring still to FIG. 3 and also to FIGS. 9, 10 and 11, the agitator driving device 10 will now be described in more detail.

The agitator driving device 10 includes a generally flat case 70 having upper and lower faces 71, 72. A set of drive heads 73 project from the case 70 through the lower face 72 which engage the tips 64 of the shafts 62 of the agitators 8.

The case 70 is formed from clear polymethylmethacrylate (or “acrylic glass”). However, other suitable plastic materials may be used, for example, plastic materials which are strong and hard enough to resist wear and use. The drive heads 73 are formed from polyoxymethylene. Other suitable plastic materials may be used, for example, plastic materials which are strong and hard enough to resist wear and use

The case 70 generally comprises upper and lower parts 74, 75. The lower part 74 (herein also referred to as a “main part”) has an upper face 76. The upper part 75 (herein also referred to as a “lid”) sits on the upper face 76 of the lower part 74.

The lower part 74 comprises a base portion 77 having a periphery 78 and a wall portion 79, which has an inner periphery 8o and an outer periphery 81. The base periphery 78 and the outer wall periphery 81 are generally co-extensive. The base portion 77 and the wall portion 79 define a recess 82. Thus, when the upper case part 75 is placed on the lower part 74, a cavity 83 is formed.

The case 70 houses a gear train 84 which includes a drive gear 85 having a coupling 86 for receiving drive from the motor 12, a set of idler gears 87 and a set of driven gears 88. The gears 85, 87, 88 take the form of external spur gears each having a stub 89, 90. The drive gear 85 and the driven gears 88 are arranged in a rectangular array and are coupled to an adjacent driven gear 88 in the same row 91 by idler gears 84 which are also arranged in a rectangular array. A pair of idler gears 84 also couple the drive gear 85 and adjacent driven gears 88 in the same column 92. The drive gear 85 and each driven gear 88 are attached to respective drive heads 73.

The upper case part 75 includes a through hole (not shown) which allows a drive shaft (not shown) from the motor 12 to couple to the drive gear 85. The lower case part 75 includes a set of through holes 93 through which the drive heads 73 project.

The gear train 84 is sandwiched between the lower and upper case parts 74, 75.

The lower case part 74 includes blind holes 94 for receiving idler gears shafts 95. The upper case part 75 includes blind holes 97 for receiving idler gear shafts 95 and driven gear shafts 98. Each driven gear 88 is provided with lower and upper bushes 99, 100.

The gear train 84 lie in the same level. However, a multi-level arrangement can be used. Arrangements using dual spur gears can be used. Other types of gears can also be used.

Each drive head 73 includes a conical recess 101 and a centrally-aligned axial slot 101 for the receiving tips 64 of the agitator shafts 62. The conical recess 101 helps to seat a tip 64 and guide it into the slot 102.

The gear train 84 causes the drive heads 73 to rotate at the same rate and in the same sense (i.e. angular direction). This can help to ensure that mixing is the same in each bottle 6.

The case 70 can help to avoid clothing or a part of the body (such as finger) being pulled into the gear train 84. Furthermore, by forming the case 70 from a transparent material, a visual inspection of the agitator driving device 10 can be used to confirm that all the agitators 8 are being driven and being driven at the same speed.

As explained earlier, the driving device 10 is held above the water bath 2 by posts 11. Each post 11 is secured to the lid 4 and case 70 by first and second bolts 103, 104.

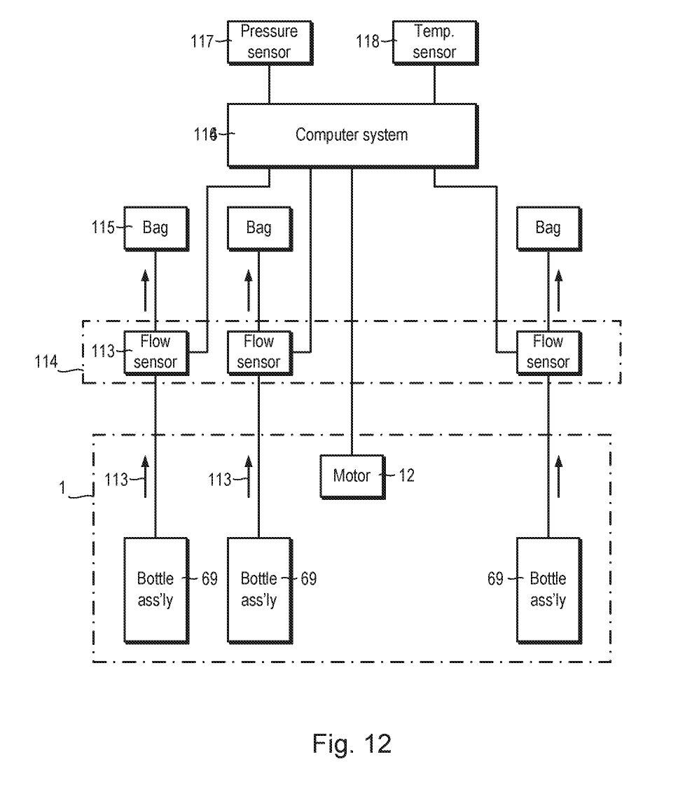

Referring to FIG. 12, a biogas potential (RBP) testing system 111 is shown which includes the static batch rector system 1 hereinbefore described.

The system 111 includes a set of flow sensors 112 which receives gas 113 from the bottle assemblies 18.

Each flow sensor 112 can take the form of a gas tumbler having a housing which contains a pivoted trapezoidal block and which is partially filled with water. As gas 113 enters the tumbler, it is trapped under the block which is initially in a rest position. A volume of gas begins to collect and starts to lift one side of the block. This continues until a sufficiently large volume of gas has been collected which tilts the block enough to allow the volume of gas to escape. The block returns to its rest position (i.e. it tumbles) and the process is repeated. The volume of gas needed to tip the block is known and by counting each time the block tumbles (for example using a magnet connected to the block and a reed switch), the total volume of gas can be calculated.

The flow sensors 112 may be housed in a single block 114.

Gas may be collected in bag 115 or other collection system for analysis.

Each bottle assembly 18 is provided with its own flow sensor 112 which is cooperatively connected to a computer system 116. To maintain accurate measurement, the ambient temperature and atmospheric pressure can be recorded using sensors 117, 118.

The computer system 116 controls operation of the motor 12. The motor 12 may be driven at a constant speed or may be driven at a speed and, if necessary stopped, according to a time-varying speed profile. The motor 12 may be driven in different directions, i.e. clockwise or anti-clockwise.

The motor 12 has sufficient power to allow agitation of liquids (which may include solids) having a high dry solids content, for example, up to 40% or more.

It will be appreciated that many modifications may be made to the embodiments hereinbefore described.

The reactors need not be bottle-shaped or be made from plastic. The reactor can be any suitable form of vessel, may be formed from a suitable material, such as metal, and may be take a suitable size or shape. Thus, a reactor can take the form of stainless steel vessel.

The cap and bottle may be secured by an arrangement other than screw engagement. For example, the cap may push-fit onto the neck of the bottle and may be held using one or more clips, tape or other securing means.

1. A device for driving a plurality of rotatable agitators for use in a static batch reactor system, the device comprising:
a planar case having first and second opposite faces;
a plurality of drive heads projecting from the case through the second face; and
a gear train protectively housed in the case, the gear train comprising a driver gear having a coupling for receiving drive from a motor, a set of idler gears and a set of driven gears, each driven gear attached to a respective drive head for driving a rotatable agitator, the gear train configured to cause the drive heads to rotate at the same rate.

2. The device according to claim 1, wherein the gear train is configured to cause the drive heads to rotate in the same angular direction.

3. The device according to claim 1, wherein the drive heads are arranged in an array.

4. The device according to claim 3, wherein the array is a rectangular array.

5. The device according to claim 3, wherein the drive heads comprise at least 3 drive heads arranged spaced along a first direction and at least 3 drive heads spaced along a second direction orthogonal to the first direction.

6. The device according to claim 1, wherein the case comprises:
a main part having a main recess for accommodating the receiving the gears and further recesses for accommodating shafts on a first face of the gears; and
a lid portion having recesses for accommodating shafts on a second, opposite face of the gears.

7. The device according to claim 1, further comprising:
one or more supports for fixing the device to a bath.

8. The device according to claim 1, further comprising:
a motor coupled to the driver gear.

9. The device according to claim 8, wherein the motor is disposed on the first face of the case.

10. The device according to claim 1, wherein each drive head includes a seat for receiving a drive shaft of a rotatable agitator.

11. The device according to claim 10, wherein each seat comprises a conical recess and a centrally aligned axially extending slot.

12. A cap which comprises:
a main body comprising:
a top portion having a center and a periphery; and
an annular skirt depending downwardly from the periphery of the top portion having an internal screw thread for engaging an external screw thread of a bottle;
an elongate neck extending upwardly from the center of the top portion along a central axis to a first distal end having an axial recess;
an elongate insert depending downwardly from the top potion of the main body along the central axis to a second, opposite distal end;
a passage extending along the central axis from the axial recess to the second distal end though the neck, main body and insert, the passage including at a first section which is relatively narrow and a second section disposed between the first section and the recess which is relatively wide; and
a duct extending through the top portion from a pipe connector on a topside of the top portion to an underside.

13. A cap assembly for a static reactor bottle, the cap assembly comprising:
the cap according to claim 12; and
an agitator comprising a shaft disposed in the passage and a paddle.

14. The cap assembly according to claim 13, wherein the insert is removably attached to the main portion.

15. The cap assembly according to claim 13, wherein the duct is inclined with respect to the central axis.

16. The cap assembly according to claim 13, further comprising:
a further passage extending through the top portion from the underside to the topside.

17. The cap assembly according to claim 16, wherein the further passage runs parallel to the passage.

18. The cap assembly according to claim 16, further comprising a tube passing through or in fluid communication with the further passage depending downwardly from the main body of the cap.

19. A first bottle assembly comprising:
a bottle comprising a body and a neck having an external screw thread; and
the cap assembly according to claim 13 which is screw engaged with the bottle.

20. A bottle assembly comprising:
a bottle comprising a body and a neck having an external screw thread; and
the cap assembly according to claim 18 which is screw engaged with the bottle,
wherein the tube has a distal end which lies at a level at or below a mid-point between top and bottom of an inside of the bottle.

21. A static batch reactor system comprising:
a bath;
the first bottle assembly according to claim 19 at least partially immersed in water in the bath;
a second bottle assembly according to claim 19 at least partially immersed in water in the bath; and
the device according to claim 1, wherein each agitator is coupled to a respective drive head.

22. The system according to claim 21, further comprising:
at least one gas flow measuring device, each of the pipe connectors included in the first and second bottle assemblies in fluid communication with a respective gas flow measuring device.

23. The system according to claim 21, wherein the system is arranged for testing residual biogas potential.

24. A method comprising:
adding feedstock to at least two bottles;
assembling at least two bottle assemblies according to claim 19;
placing the at least two bottle assemblies in a bath;
fitting the device according to claim 1 wherein each agitator is coupled to a respective drive head; and
applying power to a motor coupled to the driver gear.

25. The method according to claim 24, further comprising:
providing a flow sensor for each of the bottle assemblies.

26. A method of using the static batch reactor system according to claim 21, the method comprising:
driving the device.
